(19) United States
(12) Patent Application Publication
(10) Pub. No.: US 2025/0114132 A1
(43) Pub. Date: Apr. 10, 2025

(54) ANATOMICALLY SHAPED INLAY STEMLESS SHOULDER IMPLANT

(71) Applicant: Zimmer, Inc., Warsaw, IN (US)

(72) Inventors: Yang Wook Son, Granger, IN (US); Christine Mirja Mueri, Winterthur (CH); Lori Lynn Bowman, Churubusco, IN (US)

(21) Appl. No.: 18/889,792

(22) Filed: Sep. 19, 2024

Related U.S. Application Data
(60) Provisional application No. 63/589,243, filed on Oct. 10, 2023.

(51) Int. Cl. A61B 17/84; A61F 2/30; A61F 2/40

(52) U.S. Cl. CPC A61B 17/844; A61F 2/40; A61F 2002/30884

(57) ABSTRACT

A stemless implant configured to be inserted into a bone of a patient can include a bone anchor and an attachable component. The bone anchor can be configured to be mounted to the bone of the patient. The bone anchor can include a central hub and a plurality of fins. The central hub can define an aperture. The plurality of fins can be arranged in an asymmetric pattern. Each fin of the plurality of fins can include a proximal surface including a curvature such that a radial end of the respective fin of the plurality of fins extends proximally beyond the central hub. The attachable component can be configured to be inserted into the aperture of the central hub.

### CLAIM OF PRIORITY

This application claims the benefit of U.S. Provisional Patent Application Ser. No. 63/589,243, filed on Oct. 10, 2023, the benefit of priority of which is claimed hereby, and which is incorporated by reference herein in its entirety.

### TECHNICAL FIELD

Examples described herein generally relate to an orthopedic system. More specifically, examples described herein relate to a shoulder implant system.

### BACKGROUND

In a healthy shoulder, the proximal humerus is generally ball-shaped and articulates within a socket formed by the scapula, called the glenoid, to form the shoulder joint. Some implant systems for the total replacement of the shoulder joint generally replicate the natural anatomy of the shoulder. Such implant systems can include a humeral component having a stem that fits within the humeral canal and an articulating head that articulates within the socket of a glenoid component implanted within the glenoid of the scapula. Reverse-type shoulder implant systems have been developed in which the conventional ball-and-socket configuration, which replicates the natural anatomy of the shoulder, is reversed. For example, a reverse-type shoulder implant system can include a concave recessed articulating component at the proximal end of the humeral component, which can articulate against a convex portion of a glenosphere of a glenoid component.

The design of humeral implants for shoulder arthroplasty has evolved over the years. Whether standard or reverse, the humeral component of traditional shoulder arthroplasty is traditionally designed with a stemmed implant. However, complications with stemmed implants have been reported, such as periprosthetic humeral fractures after shoulder arthroplasty and intra-operative difficulties during revision surgeries. Accordingly, stemless shoulder prosthetics have been introduced. The key feature of a stemless humeral anchor implant is a metaphyseal fixation, which can reduce potential risks associated with using a stemmed humeral implant. However, current stemless humeral anchor systems do not account for all patient populations because size or shape is not designed anatomically to account for the asymmetry of proximal humeral bones where the dense bone is located toward the cortical bone. Moreover, currently available humeral anchor systems do not convertibility from anatomic shoulder arthroplasty to reverse shoulder arthroplasty.

### SUMMARY

In examples, a stemless implant configured to be inserted into a bone of a patient can include a bone anchor and an attachable component. The bone anchor can be configured to be mounted to the bone of the patient. The bone anchor can include a central hub and a plurality of fins. The central hub can define an aperture. The plurality of fins can be arranged in an asymmetric pattern. Each fin of the plurality of fins can include a proximal surface including a curvature such that a radial end of the respective fin of the plurality of fins extends proximally beyond the central hub. The attachable component can be configured to be inserted into the aperture of the central hub.

In examples, a system configured to be implanted into a patient can include a first anchor, a first component, a second anchor, and a second component. the first anchor can be configured to be selectively mounted to a first bone. The first component can be configured to be mounted to the first anchor such that the first component is positioned proximal to a resected surface of a first bone of the patient. The second anchor can be configured to be selectively mounted to the first bone. The second component can be configured to be mounted to the second anchor such that the second component is positioned distal to the resected surface of the first bone. Any one of the first anchor and the first component or the second anchor and the second component can be selected intraoperatively to be inserted into the bone of the patient.

In examples, a method of implanting an implant can include installing a bone anchor into a bone of a patient. The bone anchor can include a central hub, and a plurality of fins. The hub can define an aperture. The plurality of fins can extend radially outward from the central hub and be arranged in an asymmetric pattern. Each fin of the plurality of fins can include a proximal surface including a curvature such that a radial end of the respective fin of the plurality of fins is proximally above the central hub. The method can also include installing an attachable component to the aperture of the bone anchor.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 shows an example of an onlay arthroplasty system for an anatomic shoulder implant.

FIG. 2 shows an example of an onlay arthroplasty system for a reverse shoulder implant.

FIG. 3 shows an example of an inlay arthroplasty system for a reverse shoulder implant.

FIG. 4 shows a cross-sectional view of an example of an inlay arthroplasty system for a reverse shoulder implant.

FIG. 5 shows a top view of an example of an inlay arthroplasty system.

FIG. 6 shows a side view of an example of an inlay arthroplasty system.

FIG. 7 shows a cross-sectional view taken along line 7-7 from FIG. 5 of an example of an inlay arthroplasty system.

FIG. 8 shows an example arthroplasty system including inlay and onlay shoulder implants.

FIG. 9 shows a schematic view of an example method.

### DETAILED DESCRIPTION

As used herein, the following directional definitions apply. Anterior and posterior mean nearer the front or nearer the rear of the body, respectively, proximal, and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

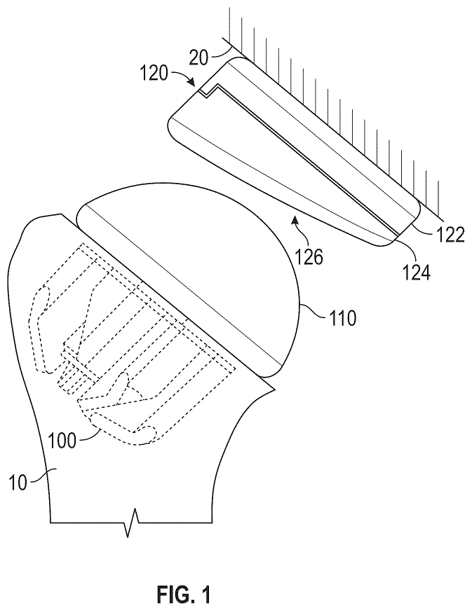

FIG. 1 shows an arthroplasty system for an anatomic shoulder implant, in accordance with one embodiment. In this example, the system can include an anchor 100 configured to be mounted to a humerus 10. The anchor 100 can be configured to selectively receive an attachable component. In examples, a humeral component, such as a humeral head 110 can be mounted to the anchor 100. As will be further discussed below, the humeral head 110 can include a post or cone, which can include a male taper extending from a bottom surface. The post or cone can mount to a cylindrical hole in the anchor 100.

The system can also include a complimentary component, such as a concave recessed articulating component 120, which can include a tray 122 and a bearing 124. The bearing 124 can include a concave bearing surface 126, which can articulate with the humeral head 110 after the implantation. The articulating component 120 can be configured to be mounted to a scapula 20 proximate to a glenoid cavity.

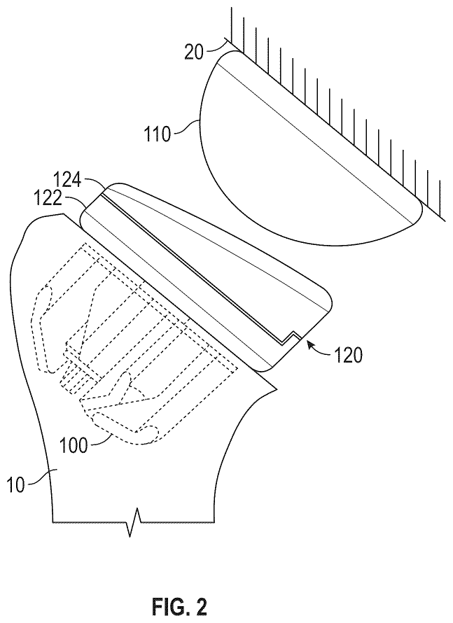

FIG. 2 shows an arthroplasty system for a reverse shoulder implant. Here, the glenosphere can be mounted to the scapula 20. As discussed above, the anchor 100 can be configured to selectively receive an attachable component. In examples, the articulating component 120 can be mounted to the humerus 10 via the anchor 100. Again, a post can extend from a bottom of the tray 122 and can be mounted to a hole located in the anchor 100.

As noted above, currently marketed stemless humeral anchor implants do not account for all patient population because the sizes and shapes of the currently marketed stemless humeral anchors are not designed anatomically to account for asymmetry of proximal humeral bones where dense bone is located toward the cortical bone. Moreover, the currently available stemless humeral anchor systems do not offer convertibility from anatomic shoulder arthroplasty to reverse shoulder arthroplasty.

Accordingly, the present system can provide the anchor 100 designed for anatomical shape and fixation stability. In examples, the design of the anchor 100 can be determined using methods described in U.S. application Ser. No. 18/108,885, filed Feb. 13, 2023, which is incorporated herein by reference in its entirety. Moreover, the present system can also allow a single humeral anchor system to be used for either anatomic or reverse total shoulder arthroplasty. The anchor 100 can be used as a primary reverse shoulder arthroplasty or convertible intra-operatively or in a revision situation.

The onlay system shown in FIGS. 1 and 2 can be designed as described in U.S. application Ser. No. 18/227,664, filed Jul. 28, 2023, which is incorporated herein by reference in its entirety. Thus, as shown in FIGS. 1 and 2, the anchor 100 can have a superior surface that is substantially parallel to the resected surface of the bone of the patient when the anchor 100 is installed within the bone of the patient. As discussed herein, the anchor 100 can be configured to be inserted below the resected surface of the bone such that the humeral head 110 or the articulating component 120 attached to the anchor 100 can be adjacent the resected surface when installed into the anchor 100. As such, the onlay system can include a lowest point of articulation (e.g., a closest point that a complementary component (the articulating component 120 for a reverse shoulder arthroplasty) can be to the resected surface of the bone during the articulation of the components between 4.5 mm and 12.5 mm above the resected surface of the bone. In examples, the onlay system can include a lowest point of articulation between 2 mm and 14 mm above the resected surface of the bone.

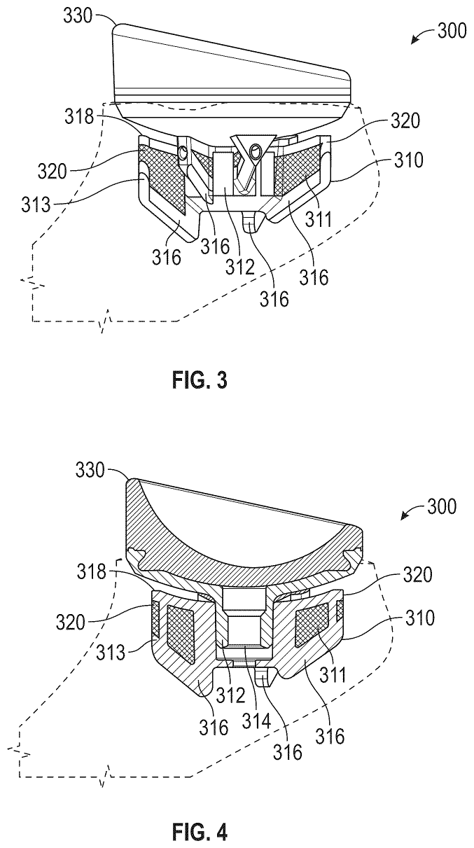

As discussed above, the anchor 100 can be an onlay anchor, which can be installed below the resected surface of the bone such as to support an attachable component adjacent and proximal to the resected surface (as shown in FIGS. 1 and 2). In examples, the anchor can also be an inlay anchor, which can be seated below a resected surface of the bone such as to support the attachable component adjacent to and distal to the resected surface. Such examples of an anchor will be discussed herein with reference to FIGS. 3-7.

FIGS. 3 and 4 will be discussed together below. FIG. 3 shows an example of an inlay arthroplasty (inlay system 300) for a shoulder implant. FIG. 4 shows a cross-sectional view of an example of the inlay system 300. The inlay system 300 can be configured to be inserted into the bone of a patient. The inlay system 300 can include a bone anchor 310 and an attachable component 330.

The bone anchor 310 can be configured to be mounted to the bone of the patient. In examples, the bone anchor 310 can be mounted to the humeral bone of the patient. In examples, the bone anchor 310 can include portions that include porous biocompatible materials, such as, for example, OsseoTi®, or the like, which can promote bone growth to help support the inlay system 300 after implantation into the bone of the patient. In examples, the portions of porous biocompatible materials can be internal portions 311, which can be surrounded by outer sections 313 including another material, such as, for example, titanium, aluminum, stainless steel, any other biocompatible material, any combination or alloy thereof, or the like. The bone anchor 310 can include a central hub 312 and a plurality of fins 316.

The central hub 312 can define an aperture 314 (shown in FIG. 4). The plurality of fins 316 can extend radially from the central hub 312. The plurality of fins 316 can be arranged in an asymmetric pattern. Each fin of the plurality of fins 316 can include a proximal surface 318. The proximal surface 318 can include a curvature such that a radial end 320 of the respective fin of the plurality of fins 316 extends proximally beyond the central hub 312.

The attachable component 330 can be configured to be inserted into the aperture 314 of the central hub 312. In examples, the attachable component 330 can include a humeral head configured to be mounted to the bone anchor 310. In examples, the attachable component 330 can include a concave recessed articulating component, including a humeral tray and a bearing assembly, such as the example shown in FIGS. 3 and 4. In examples, the central hub 312 can be configured to receive the attachable component 330, which can be a humeral head or a humeral tray.

As shown in FIGS. 3 and 4, the bone anchor 310 can be configured to support the attachable component 330 adjacent and distal to the resected surface of the bone. As such, the bone anchor 310 can permit the inlay system 300 to have a lower lowest point of articulation than the onlay system (shown in FIGS. 1 and 2). For example, the inlay system 300 can have a lowest point of articulation that ranges from 1.5 mm below the resected surface of the bone and 6.5 mm above the resected surface of the bone. In examples, the inlay system 300 can have a lowest point of articulation that ranges from 4 mm below the resected surface of the bone to 10 mm above the resected surface of the bone.

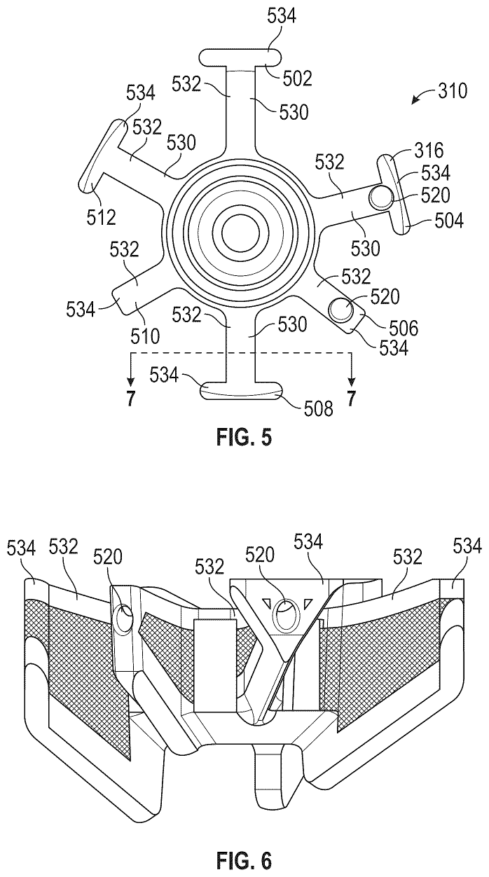

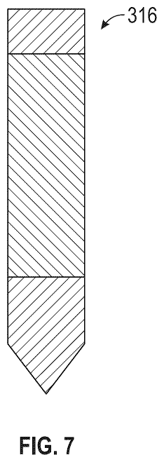

FIGS. 5-7 will be discussed together below. FIG. 5 shows a top view of an example of the bone anchor 310 of the inlay system 300 (FIG. 3). FIG. 6 shows a side view of an example of the bone anchor 310 of the inlay system 300. FIG. 7 shows a cross-sectional view taken along line 7-7 from FIG. 5 of an example of the bone anchor 310 of the inlay system 300.

As shown in FIGS. 5-7, the plurality of fins 316 can be sized and positioned to conserve bone, such as, for example, the humeral bone of the patient. For example, as shown in FIGS. 6 and 7, one or more fins of the plurality of fins 316 can include a pointed portion, or a v-shaped portion, that forms an anterior surface of the bone anchor 310. The V-shaped portion can help with the insertion of the bone anchor 310 into the bone without requiring more bone being removed than necessary. The V-shaped portion can also help provide a cutting edge for the bone anchor 310 such as to aid in the insertion of the bone anchor 310 into the bone. In examples, the V-shaped portion can allow for self-punching during insertion of the bone anchor 310 into the bone of the patient.

In examples, the plurality of fins 316 can include between two and ten fins. In examples, the plurality of fins 316 can include between 4 and 7 fins. For example, the plurality of fins 316 can include six fins, such as, for example, a first fin 502, a second fin 504, a third fin 506, a fourth fin 508, a fifth fin 510, and a sixth fin 512. The first fin 502 can be configured to be proximate to a greater tubercle of the humerus. The second fin 504 can be configured to be positioned proximate to a lesser tubercle of the humerus. The fourth fin 508 can be positioned 180 degrees from the first fin 502. The third fin 506 can be between the second fin 504 and the fourth fin 508. For example, the third fin 506 can be centered between the second fin 504 and the fourth fin 508. The fifth fin 510 can be positioned such that there is an equal angle between the third fin 506 and the fourth fin 508 and between the fourth fin 508 and the fifth fin 510. The sixth fin 512 can be between the fifth fin 510 and the first fin 502. For example, the sixth fin 512 can be centered between the fifth fin 510 and the first fin 502. Two or more of the fins of the plurality of fins 316 can include suture holes 520. The suture holes 520 can be configured to receive a suture. Each fin (e.g., the first fin 502, the second fin 504, the third fin 506, the fourth fin 508, the fifth fin 510, and the sixth fin 512) can have a different radial length extending from the central hub 312 and can have different heights extending from the proximal surface 318 to a bottom surface of the respective fin. The bottom edge of each of the fins can include a sloped portion such that the portion of the fin adjacent the central hub 312 is lower than an outer tip of the fin so that the bottom edge slopes upward from the central hub 312.

In examples, one or more of the fins of the plurality of fins 316 can include a T-shaped cross section 530 having a first portion 532 and a second portion 534. The first portion 532 can extend from the central hub 312 and the second portion 534 can extend perpendicular to the first portion 532. The T-shaped cross section 530 can help stabilize the bone anchor 310 within the bone of the patient as the first portion 532 increases a contact surface area between the bone anchor 310 and the bone of the patient. In examples, one or more of the fins of the plurality of fins 316 can be straight, such as, for example, they do not include the T-shaped cross section 530. The fins of the plurality of fins 316 that have a straight profile can help reduce impingement on the cortical bone of the patient.

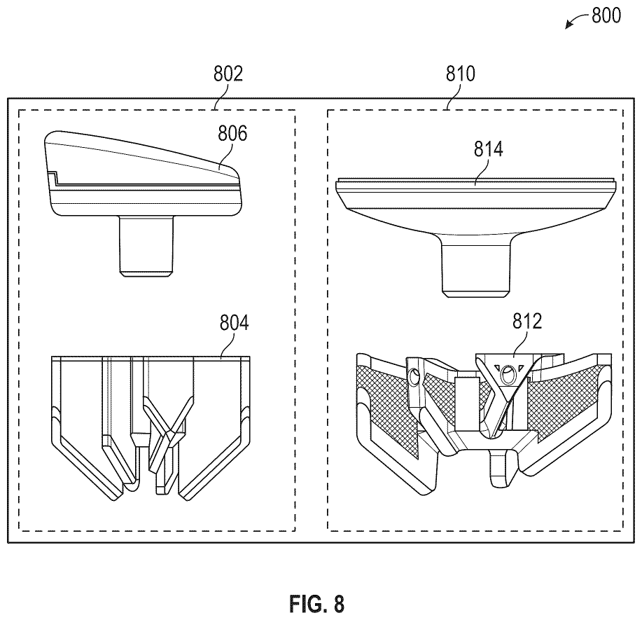

FIG. 8 shows an example arthroplasty system 800. The system 800 can be configured to be implanted into the patient. The system 800 can be shipped to a medical professional such that the medical professional can preoperatively, or intraoperatively, select an onlay system (e.g., the anchor 100 and articulating component 120) or an inlay system (e.g., the inlay system 300 including the bone anchor 310 and the attachable component 330). In examples, the system 800 can include a plurality of onlay systems 802 and a plurality of inlay systems 810.

The plurality of the onlay systems 802 can be configured to be inserted into the bone of the patient above the resected surface of the bone. Each onlay system of the plurality of onlay systems 802 can include a first anchor 804 and a first component 806. The first anchor 804 can be configured to be selectively mounted to a first bone of the patient. The first component 806 can be configured to be mounted to the first anchor 804 such that the first component 806 is positioned proximal to the resected surface of the first bone of the patient.

The plurality of the inlay systems 810 can be configured to be inserted into the bone of the patient such that at least a portion of the plurality of inlay systems 810 is below the resected surface of the bone. Each inlay system of the plurality of inlay systems 810 can include a second anchor 812 and a second component 814. The second anchor 812 can be configured to be selectively mounted to the first bone of the patient. The second component 814 can be configured to be mounted to the second anchor 812 such that the second component 814 is positioned distal to the resected surface of the first bone of the patient. Any onlay system of the plurality of onlay systems 802 including the first anchor 804 and the first component 806 or the plurality of inlay systems 810 including the second anchor 812 and the second component 814 can be selected intraoperatively to be inserted into the bone of the patient.

Each onlay system of the plurality of onlay systems 802 can be a different size, which can be selected by the surgeon based on the geometric size of the bone of the patient. As discussed herein, the onlay system of varying sizes can include a lowest point of articulation between, for example, 4 mm and 13 mm above the resected surface of the bone. More specifically, the onlay system of varying sizes can include a lowest point of articulation between, for example, 4.5 mm and 12.5 mm. In examples, the onlay system of varying sizes can include a lowest point of articulation between, for example, 3 mm and 14 mm.

Each inlay system of the plurality of inlay systems 810 can be a different size, which can be selected by the surgeon based on the geometric size of the bone of the patient. As discussed herein, the inlay system of varying sizes can include a lowest point of articulation between, for example, 2 mm below the resected surface and 7 mm above the resected surface of the bone. More specifically, the inlay system of varying sizes can include a lowest point of articulation between, for example, 1.5 mm below the resected surface of the bone and 6.5 mm above the resected surface of the bone. In examples, the inlay system of varying sizes can include a lowest point of articulation between, for example, 4 mm below the resected surface of the bone and 8 mm above the resected surface of the bone.

Therefore, the system 800, which can include the plurality of onlay systems 802 and the plurality of inlay systems 810, can have a lowest point of articulation range between 4 mm below the resected surface and 14 mm above the resected surface. In examples, the lowest point of articulation of the system 800 can range between 1.5 mm below the resected surface and 12.5 mm above the resected surface. In examples, the lowest point of articulation of the system 800 can range between 2 mm below the resected surface and 13 mm above the resected surface.

The system 800 can also include a plurality of complementary components (e.g., the humeral head 110 or the articulating component 120 (both from FIG. 1)), which can be used complementary to the plurality of onlay systems 802 or the plurality of inlay systems 810. The complementary components can be configured to be mounted to a second bone of the patient. The first bone and the second bone can form a joint of the patient, such as, for example, a shoulder joint or knee joint of the patient. In examples, the complementary components can be sent to the medical professionals separately from the system 800.

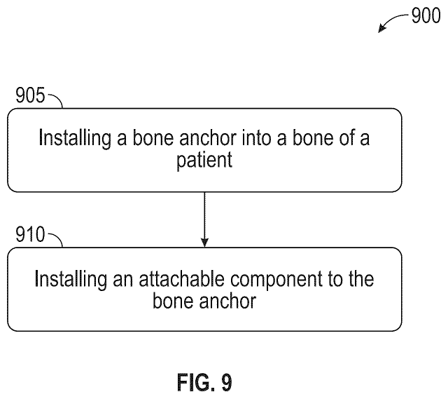

FIG. 9 shows a schematic view of an example method 900. The example 900 can be an example method for implanting an implant system (e.g., the onlay system shown in FIGS. 1 and 2 or the inlay system shown in FIGS. 3-7).

At operation 905, the method 900 can include installing a bone anchor into a bone of a patient. In examples, the bone anchor can be the anchor 100 (FIG. 1) or the bone anchor 310 (FIG. 3), which can include a central hub (e.g., the central hub 312 (FIG. 3)), and a plurality of fins (e.g., the plurality of fins 316 (FIG. 3)). Each fin of the plurality of fins can include a proximal surface including a curvature such that a radial end of the respective fin of the plurality of fins is proximally above the central hub. The central hub can define an aperture (e.g., the aperture 314 (FIG. 3)). The plurality of fins can extend radially outward from the central hub and can be arranged in an asymmetric pattern.

To install the bone anchor, the bone will need to be prepared, such as, for example, reamed, bored, broached, or the like and resected such that a distal edge of the bone can include a flat surface that can receive the bone anchor. As discussed herein, the bone anchor can be designed such as to help with the insertion of the bone anchor and to reduce an amount of bone required to be removed to insert the bone anchor.

At operation 910, the method 900 can include installing an attachable component (e.g., the humeral head 110 (FIG. 1) the articulating component 120 (FIG. 1) or the attachable component 330 (FIG. 3)) to the aperture of the bone anchor. As discussed herein, the attachable component can be installed adjacent and proximal to the resected surface of the bone for an onlay system and adjacent and distal to the resected surface of the bone for an inlay system.

The following, non-limiting examples, detail certain aspects of the present subject matter to solve the challenges and provide the benefits discussed herein, among others.

Example 1 is a stemless implant that can be configured to be inserted into a bone of a patient, and the stemless implant can include: a bone anchor configured to be mounted to the bone of the patient, the bone anchor comprising: a central hub defining an aperture; and a plurality of fins extending radially from the central hub, the plurality of fins arranged in an asymmetric pattern, each fin of the plurality of fins comprising: a proximal surface including a curvature such that a radial end of the respective fin of the plurality of fins extends proximally beyond the central hub; and an attachable component configured to be inserted into the aperture of the central hub.

In Example 2, the subject matter of Example 1 can include, wherein the attachable component can include a humeral head configured to be mounted to the bone anchor.

In Example 3, the subject matter of Examples 1-2 can include, wherein the attachable component can include a concave recessed articulating component including a humeral tray and bearing assembly.

In Example 4, the subject matter of Examples 1-3 can include, wherein the bone anchor can include a porous metal.

In Example 5, the subject matter of Examples 1-4 can include, wherein the plurality of fins are sized and positioned to conserve humeral bone.

In Example 6, the subject matter of Examples 1-5 can include, wherein the attachable component is a humeral head or a humeral tray, and wherein the bone anchor is configured to selectively receive either the humeral head or the humeral tray.

In Example 7, the subject matter of Examples 1-6 can include, wherein the plurality of fins can include six fins.

In Example 8, the subject matter of Example 7 can include, wherein the bone is a humerus, and wherein the six fins comprise: a first fin configured to be positioned proximate to a greater tubercle of the humerus; a second fin configured to be positioned proximate to a lesser tubercle of the humerus; a fourth fin positioned one hundred and eighty degrees from the first fin; a third fin centered between the second fin and the fourth fin; a fifth fin positioned such that there is an equal angle between the third fin and the fourth fin and the fourth fin and the fifth fin; and a sixth fin centered between the fifth fin and the first fin.

In Example 9, the subject matter of Examples 7-8 can include, wherein two or more fins of the plurality of fins include suture holes configured to receive a suture.

In Example 10, the subject matter of Examples 1-9 can include, wherein one or more fins of the plurality of fins include a T-shaped cross-section having a first portion extending from the central hub and a second portion perpendicular to the first portion.

Example 11 is a system configured to be implanted into a patient, the system comprising: a first anchor configured to be selectively mounted to a first bone; a first component configured to be mounted to the first anchor such that the first component is positioned proximal to a resected surface of a first bone of the patient; a second anchor configured to be selectively mounted to the first bone; and a second component configured to be mounted to the second anchor such that the second component is positioned distal to the resected surface of the first bone; wherein any one of the first anchor and the first component or the second anchor and the second component are selected intraoperatively to be inserted into the bone of the patient.

In Example 12, the subject matter of Example 11 can include, a complementary component configured to be mounted to a second bone of the patient, the second bone forming a joint with the first bone; wherein the first component and the second component are both adapted to operate with the complementary component.

In Example 13, the subject matter of Examples 11-12 can include, wherein the first anchor comprises: a central hub defining a aperture; and a plurality of fins extending radially from the central hub, the plurality of fins arranged in an asymmetric pattern and having varying heights and radial lengths.

In Example 14, the subject matter of Example 13 can include, wherein the second anchor comprises: a central hub defining a aperture; and a plurality of fins extending radially from the central hub, the plurality of fins arranged in an asymmetric pattern and having varying heights and radial lengths, each fin of the plurality of fins comprising: a proximal surface including a curvature such that a radial end of the respective fin of the plurality of fins is proximally above the central hub.

In Example 15, the subject matter of Example 14 can include, wherein the plurality of fins of the first anchor and the plurality of fins of the second anchor each comprises six fins.

In Example 16, the subject matter of Example 15 can include, wherein the bone is a humerus, and wherein the six fins of the plurality of fins of the first anchor and the plurality of fins of the second anchor each comprises: a first fin configured to be positioned proximate to a greater tubercle of the humerus; a second fin configured to be positioned proximate to a lesser tubercle; a fourth fin positioned one hundred and eighty degrees from the first fin; a third fin centered between the second fin and the fourth fin; a fifth fin positioned such that there is an equal angle between the third fin and the fourth fin and the fourth fin and the fifth fin; and a sixth fin centered between the fifth fin and the first fin.

In Example 17, the subject matter of Examples 14-16 can include, wherein two or more fins of the plurality of fins of the first anchor and two or more fins of the plurality of fins of the second anchor include suture holes configured to receive a suture.

In Example 18, the subject matter of Examples 14-17 can include, wherein one or more fins of the plurality of fins of the first anchor and one or more fins of the plurality of fins of the second anchor include a T-shaped cross-section having a first portion extending from the central hub and a second portion perpendicular to the first portion.

In Example 19, the subject matter of Examples 14-18 can include, a plurality of first anchors, each first anchor of the plurality of first anchors having a different size than other first anchors of the plurality of first anchors; and a plurality of second anchors, each second anchor of the plurality of second anchors having a different size than other second anchors of the plurality of second anchors.

Example 20 is a method of implanting an implant, the method comprising: installing a bone anchor into a bone of a patient, the bone anchor comprising: a central hub defining an aperture; and a plurality of fins extending radially outward from the central hub, the plurality of fins arranged in an asymmetric pattern, each fin of the plurality of fins comprising: a proximal surface including a curvature such that a radial end of the respective fin of the plurality of fins is proximally above the central hub; and installing an attachable component to the aperture of the bone anchor.

Example 21 is an apparatus comprising means to implement of any of Examples 1-20.

Example 22 is a system to implement any of Examples 1-20.

Example 23 is a method to implement any of Examples 1-20.

Example 24 is an apparatus, a system, or a method of any element of any of Examples 1-20.

The above-detailed description can include references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

1. A stemless implant configured to be inserted into a bone of a patient, the stemless implant comprising:
a bone anchor configured to be mounted to the bone of the patient, the bone anchor comprising:
a central hub defining an aperture; and
a plurality of fins extending radially from the central hub, the plurality of fins arranged in an asymmetric pattern, each fin of the plurality of fins comprising:
a proximal surface including a curvature such that a radial end of the respective fin of the plurality of fins extends proximally beyond the central hub; and
an attachable component configured to be inserted into the aperture of the central hub.

2. The stemless implant of claim 1, wherein the attachable component includes a humeral head configured to be mounted to the bone anchor.

3. The stemless implant of claim 1, wherein the attachable component includes a concave recessed articulating component including a humeral tray and bearing assembly.

4. The stemless implant of claim 1, wherein the bone anchor includes a porous metal.

5. The stemless implant of claim 1, wherein the plurality of fins are sized and positioned to conserve humeral bone.

6. The stemless implant of claim 1, wherein the attachable component is a humeral head or a humeral tray, and wherein the bone anchor is configured to selectively receive either the humeral head or the humeral tray.

7. The stemless implant of claim 1, wherein the plurality of fins includes six fins.

8. The stemless implant of claim 7, wherein the bone is a humerus, and wherein the six fins comprise:
a first fin configured to be positioned proximate to a greater tubercle of the humerus;
a second fin configured to be positioned proximate to a lesser tubercle of the humerus;
a fourth fin positioned 180 degrees from the first fin;
a third fin centered between the second fin and the fourth fin;
a fifth fin positioned such that there is an equal angle between the third fin and the fourth fin and the fourth fin and the fifth fin; and
a sixth fin centered between the fifth fin and the first fin.

9. The stemless implant of claim 7, wherein two or more fins of the plurality of fins include suture holes configured to receive a suture.

10. The stemless implant of claim 1, wherein one or more fins of the plurality of fins include a T-shaped cross-section having a first portion extending from the central hub and a second portion perpendicular to the first portion.

11. A system configured to be implanted into a patient, the system comprising:
a first anchor configured to be selectively mounted to a first bone;
a first component configured to be mounted to the first anchor such that the first component is positioned proximal to a resected surface of a first bone of the patient;
a second anchor configured to be selectively mounted to the first bone; and
a second component configured to be mounted to the second anchor such that the second component is positioned distal to the resected surface of the first bone;
wherein any one of the first anchor and the first component or the second anchor and the second component are selected intraoperatively to be inserted into the first bone of the patient.

12. The system of claim 11, comprising:
a complementary component configured to be mounted to a second bone of the patient, the second bone forming a joint with the first bone;
wherein the first component and the second component are both adapted to operate with the complementary component.

13. The system of claim 11, wherein the first anchor comprises:
a central hub defining an aperture; and
a plurality of fins extending radially from the central hub, the plurality of fins arranged in an asymmetric pattern and having varying heights and radial lengths.

14. The system of claim 13, wherein the second anchor comprises:
a central hub defining an aperture; and
a plurality of fins extending radially from the central hub, the plurality of fins arranged in an asymmetric pattern and having varying heights and radial lengths, each fin of the plurality of fins comprising:
a proximal surface including a curvature such that a radial end of the respective fin of the plurality of fins is proximally above the central hub.

15. The system of claim 14, wherein the plurality of fins of the first anchor and the plurality of fins of the second anchor each comprises six fins.

16. The system of claim 15, wherein the bone is a humerus, and wherein the six fins of the plurality of fins of the first anchor and the plurality of fins of the second anchor each comprises:
a first fin configured to be positioned proximate to a greater tubercle of the humerus;
a second fin configured to be positioned proximate to a lesser tubercle;
a fourth fin positioned 180 degrees from the first fin;
a third fin centered between the second fin and the fourth fin;
a fifth fin positioned such that there is an equal angle between the third fin and the fourth fin and the fourth fin and the fifth fin; and
a sixth fin centered between the fifth fin and the first fin.

17. The system of claim 14, wherein two or more fins of the plurality of fins of the first anchor and two or more fins of the plurality of fins of the second anchor include suture holes configured to receive a suture.

18. The system of claim 14, wherein one or more fins of the plurality of fins of the first anchor and one or more fins of the plurality of fins of the second anchor include a T-shaped cross-section having a first portion extending from the central hub and a second portion perpendicular to the first portion.

19. The system of claim 14, comprising:
a plurality of first anchors, each first anchor of the plurality of first anchors having a different size than other first anchors of the plurality of first anchors; and
a plurality of second anchors, each second anchor of the plurality of second anchors having a different size than other second anchors of the plurality of second anchors.

20. A method of implanting an implant, the method comprising:
installing a bone anchor into a bone of a patient, the bone anchor comprising:
a central hub defining an aperture; and
a plurality of fins extending radially outward from the central hub, the plurality of fins arranged in an asymmetric pattern, each fin of the plurality of fins comprising:
a proximal surface including a curvature such that a radial end of the respective fin of the plurality of fins is proximally above the central hub; and
installing an attachable component to the aperture of the bone anchor.
